(19) United States
(12) Patent Application Publication
(10) Pub. No.: US 2006/0045848 A1
(43) Pub. Date: Mar. 2, 2006

(54) Conscience-enhancing malice-weakening gas

(76) Inventor: Bret Bernard Cummings, El Segundo, CA (US)

Correspondence Address: Bret Cummings, 808 McCarthy Ct., El Segundo, CA, 90245, US

(21) Appl. No.: 10/870,894

(22) Filed: Aug. 24, 2004

(51) Int. Cl. A61L 9/04

(52) U.S. Cl. 424/45

(57) ABSTRACT

This invention is a mixture of neuroactive substances in the form of gases, liquid aerosols, and/or solid aerosols, which act synergistically to increase the ratio of strength between the desire to do right, called conscience, and the desire to do wrong, called malice. Neurochemically, that corresponds to increasing the ratio of activity of the H1, M1, and/or M3 receptors to the activity of the 5-HT2A, 5-HT2B, and/or 5-HT2C receptors, by facilitating the activity of the former and/or suppressing the activity of the latter. This mixture is intended to be administered to humans and other vertebrate animals primarily via aerosol containers. This mixture is intended to be used primarily by the police and the military, for the functions of active or potential conflict and investigation.

### BACKGROUND OF THE INVENTION

1. Field of the Invention

neurochemistry

2. Prior Method to Satisfy the Purpose of the Invention

methods of psychological persuasion that are far more time-consuming and only somewhat effective, and are therefore not used often

### BRIEF SUMMARY OF THE INVENTION

This invention is a mixture of neuroactive substances in the form of gases, liquid aerosols, and/or solid aerosols, which act synergistically to increase the activity of the H1, M1 and/or M3 receptors and decrease the activity of the 5-HT2A, 5-HT2B, and/or 5-HT2C receptors, thereby enhancing the desire to do right, called conscience and weakening the desire to do wrong, called malice.

### DETAILED DESCRIPTION OF THE INVENTION

This invention is a mixture of gases, liquid aerosols, and/or solid aerosols, whichever state is stable at room temperature for the substances in question. The word ‘gas’ as it is used in the title of the invention, is meant as a concise inclusive term that means ‘any substance that behaves as a gas’, such that it includes actual gases as well as liquid aerosols and solid aerosols. Conscience-enhancing malice-weakening gas may also be called by the abbreviated name ‘C/M gas’, for efficiency.

The substances of the mixture are neuroactive and are intended to be inhaled by a person or persons, or an animal or animals. Specifically, the substances-act synergistically to facilitate the activity of the H1, M1 and/or M3 receptors and suppress the activity of the 5-HT2A, 5-HT2B, and/or 5-HT2C receptors, preferably acting on all of the receptors mentioned, thereby enhancing the conscience and weakening malice, as it is known that the former 3 receptors produce conscientious sensations, whereas the latter 3 receptors produce malicious sensations.

That may be done by using one or more neurochemical methods, which correspond to specific substances that use said methods. One type of substances that may be used are those that selectively agonize the H1, M1 and M3 receptors and substances that selectively antagonize the 5-HT2A, 5-HT2B, and 5-HT2C receptors. A second type of substances that may be used are those that selectively antagonize any inhibitory receptors that suppress the activity of the H1, M1, and/or M3 receptors. The M2 receptor is an inhibitory autoreceptor that suppresses the activity of the M1 and M3 receptors, and the suppression of the activity of those 2 receptors in turn decreases the activity of the H1 receptor. The polypeptide galanin suppresses the activity of the H1, M1 and M3 receptors by blocking the T-type calcium ion channels that they use and by activating inhibitory heteroreceptors. A high level of acetylcholine causes the activity of the inhibitory M4 receptors as a side effect, which, in individuals that have a large number of said receptors, significantly impairs their multitasking ability, so one or more of the substances in the mixture should antagonize the M4 receptor so as to eliminate that side effect. A third type of substances that may be used are those that interfere with enzymes that produce or degrade serotonin, acetylcholine, and the sex hormones of dihydrotestosterone, estradiol, and progesterone. A high level of serotonin is conducive to the activity of the 5-HT2 receptors. A high level of acetylcholine is conducive to the activity of the M1 and M3 receptors, and the activity of those receptors are in turn conducive to the activity of the H1 receptor, which uses the neurotransmitter histamine. A high level of any of the sex hormones of dihydrotestosterone, estradiol, or progesterone is conducive to the activity of the 5-H2 receptors. A high level of serotonin increases galanin production. A fourth type of substances that may be used are those that sensitize specific glutamate receptors that exist on the same terminals as H1, M1 and/or M3 receptors, of which piracetam is the most effective substance. As can be seen, the 4 types of substances work synergistically. The most preferable substances of this mixture are as follows:
1. the H1 agonist histaprodifen (a substance of the first type mentioned)
2. the M1/M3 agonist cevimeline (a substance of the first type mentioned)
3. the 5-HT2B antagonist SB204741 (a substance of the first type mentioned)
4. the 5-HT2A/2C antagonist ketanserin (a substance of the first type mentioned)
5. the M2/M4 antagonist AFDX384 (a substance of the second type mentioned)
6. the galanin antagonist g40 (a substance of the second type mentioned)
7. the acetylcholine esterase inhibitor galantamine (a substance of the third type mentioned)
8. the tryptophan hydroxylase inhibitor p-chlorophenylethylamine (a substance of the third type mentioned)
9. the AMPA-3 glutamate receptor sensitizer piracetam (a substance of the fourth type mentioned)

Due to the large number of different substances that constitute that mixture, it is likely that drug interactions of some kind are likely to occur when it is inhaled, although that would occur at a higher dose than that which is required for the mixture to take effect.

Method of Manufacture:

The substances of this mixture can be manufactured by the usual chemical synthesis methods that are used by pharmaceutical companies. The most common methods thereof are:
1. mixing primary component substances, initiating chemical reactions, often with a catalyst and/or heat, purifying the desired substances of that particular reaction, and then processing the resulting substances in the same manner, in succession until the desired end products are created
2. mixing primary component substances, using biological enzymes to produce specific chemical reactions, either in a succession of separate reactions and purifications, or all at once, and purifying the desired end products

The desired end products are then mixed together.

Application:

This mixture of airborne substances is intended to be used primarily by the police and military, and particularly for the functions of animal control, riot control or other mob control, halting crimes or military aggression in progress, suspect apprehension, hostage and human shield situations, and interrogation, although it may also be used for any other function for which it is useful and by any other people to whom it is useful. This mixture does not prevent suspects from fleeing from the police in cases in which the suspect has already done a significant crime, as doing so is caused by fear of punishment rather than malice or weakness of conscience. In interrogation, conscience-enhancing malice-weakening gas is especially effective in preventing acts of fraudulent accusations of criminal acts, fraudulent claims of innocence of significantly malicious criminal acts, and fraudulent characterizations of a person's character, as such criminal statements are some of the very most common criminal acts that are caused by malice. The mixture is also effective in causing interrogated suspects to admit any such deceptions that they had made earlier, if there is an agreement made beforehand to minimize the resulting punishments for said deceptions. Malicious deceivers are often identifiable by their facial expression and tone of voice, with the former being characterized by heightened eyebrows and a widened mouth, but conscience-enhancing malice-weakening gas can force such people to admit their deceptions and to state the truth.

Method of Administration:

This mixture may be conveyed to it's target in any way that is useful, such as by vials held to the noses of interrogated suspects, aerosol containers sprayed by personnel on foot, gas canisters or weighted aerosol canisters thrown by personnel on foot, gas canisters or weighted aerosol canisters launched from vehicles or tanks, or styrofoam-padded gas canisters or aerosol canisters dropped or launched from airplanes or helicopters. The substances of this mixture are most likely to be solid at room temperature, and because of that, they are most likely to be administered via aerosol containers.

1. I claim the following as my invention: any mixture of 2 or more neuroactive substances in the form of gases, liquid aerosols, and/or solid aerosols, that act synergistically to increase the ratio of strength between the desire to do right, called conscience, and the desire to do wrong, called malice. Neurochemically, that corresponds to increasing the ratio of activity of the H1, M1, and/or M3 receptors to the activity of the 5-HT2A, 5-HT2B, and/or 5-HT2C receptors, by facilitating the activity of the former and/or suppressing the activity of the latter.
